(19) United States
(12) Patent Application Publication
(10) Pub. No.: US 2024/0013908 A1
(43) Pub. Date: Jan. 11, 2024

(54) SALIVA SAMPLE TESTING DEVICES, METHODS, AND MOBILE APP

(71) Applicant: Oral Genome Corporation, Carlsbad, CA (US)

(72) Inventors: Tina Saw, Carlsbad, CA (US); Thanh Luu, Carlsbad, CA (US)

(73) Assignee: Oral Genome Corporation, Carlsbad, CA (US)

(21) Appl. No.: 18/131,523

(22) Filed: Apr. 6, 2023

Related U.S. Application Data
(63) Continuation of application No. 17/857,757, filed on Jul. 5, 2022, now abandoned.

(51) Int. Cl. G16H 40/67; G01N 33/543; G01N 33/52; G16H 50/30; G16H 30/20; G01N 33/84

(52) U.S. Cl. CPC G16H 40/67; G01N 33/54388; G01N 33/52; G16H 50/30; G16H 30/20; G01N 33/84

(57) ABSTRACT

A method of testing a saliva sample includes: providing a testing device having a color palette and multiple test areas, using colorimetric analysis and lateral flow assay tests; collecting at least a prescribed volume of a saliva sample; dispensing, onto each of multiple test areas of a testing device, a respective portion of the collected saliva sample, the testing device thereby developing a respective discernible reaction at each test area; capturing, by a computing device, an image of testing device; analyzing, through a testing platform, the image by colorimetric and liquid flow assay analysis; and displaying results of the colorimetric analysis, the displayed results representing oral health parameters. The displayed results including quantitative indices and trend identifiers related to oral health parameters. A kit for use in conducting the above method includes the sample reactive device.

### RELATED APPLICATIONS

This application claims priority to U.S. patent application Ser. No. 17,857,757, titled “SALIVA SAMPLE TESTING DEVICES, METHODS, AND MOBILE APP,” filed on Jul. 5, 2022, which claimed priority to U.S. Provisional Patent Application No. 63/277,669, titled “FLUID SAMPLE TESTING DEVICES, METHODS, AND MOBILE APP,” filed on Nov. 10, 2021, the entire contents of which are incorporated by reference herein.

### TECHNICAL FIELD

The present disclosure relates to oral health testing. More particularly, the present disclosure relates to saliva testing kits, devices, and methods for rapid oral health screening without the use of a laboratory.

### BACKGROUND

Conventional health testing typically is conducted at health-care facilities by care providers and health technicians and involves testing of blood and urine. Test results typically await laboratory analysis, which can be delayed according to the waiting times related to the volume of tests in a queue, according to the processing times of testing procedures, and according to the need for a qualified professional to inspect and interpret the results of each test. Additionally, test results can be delayed due to delivery and/or mailing of samples to a laboratory. However, even those conveniences are often unavailable as samples can be degraded due to conditions, such as the weather, and the amount of time it takes to get a sample to a laboratory. Thus, a user is typically required to travel to and appear at a facility but is not assured rapid test results regardless of whether an appointment was made well in advance.

There are at home test kits that are available by which the user can self-administer a test or collect a sample at home, usually utilizing urine, and mail the samples to a laboratory by mail. After waiting for samples to arrive and for laboratory analysis, the results are sent to the user by email or electronically on a portal.

Improvements are needed for at home testing health testing, particularly testing related to oral health, which utilizes saliva, and provides efficient and timely results.

### SUMMARY

This summary is provided to briefly introduce concepts that are further described in the following detailed descriptions. This summary is not intended to identify key features or essential features of the claimed subject matter, nor is it to be construed as limiting the scope of the claimed subject matter.

In at least one embodiment, a method of testing a saliva sample to determine oral health parameters includes: providing a testing device which has a color palette and multiple test areas, including colorimetric and lateral flow test areas which react due to attributes of a saliva sample; collecting a first prescribed volume of user saliva; dispensing a second prescribed volume of user saliva apportioned from the first volume onto each of the test areas to generate a reaction; capturing an image of the testing device including generated reactions with an imaging sensor of a computing device; analyzing the image through a testing platform accessed by the computing device, where the testing platform is configured to generate results for oral health parameters based on reactions; and displaying results generated for oral health parameters on a display module of the computing device.

The reactions may include color changes for colorimetric analysis test areas and generated indicators for lateral flow assay test areas.

A sterile collection device may be utilized to collect the first prescribed volume of user saliva and from which the second prescribed volume of user saliva may be dispensed onto each of the test areas.

The testing platform may be stored within a memory module of the computing device.

The image of the testing device including the generated reactions, such as color changes and indicators, may be stored in the memory module of the computing device.

The testing platform may calibrate an image or an analysis based on the color palette of the testing device to increase the accuracy of results generated for oral health parameters by the testing platform.

The testing platform may be stored in a network accessible memory module, such as on a cloud-based server, and be accessed by a network communication module of the computing device to access the testing platform.

The results generated by the testing platform may be stored in a memory module of the computing device with identifying time and date data. The testing platform may generate trend identifiers based on the results stored in the memory module and their respective time and date data. The trend identifiers may be displayed on a display module of the computing device.

The results and/or the trend identifiers generated by the testing platform for the oral health parameters may be sent to a remote device through a network with a network communication module of the computing device.

The results generated for oral health parameters can include quantitative indices of each of the respective oral health parameters.

The value for each of the quantitative indices may be based on, or result from, a specific color change or indicators generated in a test area.

The testing device may further comprise a casing to enhance the rigidity, durability, and longevity of the testing device and help direct saliva samples to intended test areas, preventing inadvertent dispensing in an unintended location. Cutouts in the casing provide access to dispensing portions of the test area, indications of what portions of the test areas are dispensing portions, and a view of the test area, particularly the portion where a reaction will be visible.

The testing area may comprise a dispensing portion where the sample is placed to produce a reaction and ensure accurate results.

The method step of analyzing an image with the testing platform may further comprise determining if the image has characteristics which might prevent accurate results based on the image. For example, the testing platform could determine if the image is too dark or the testing device is too far away from the imaging sensor.

The attributes of the saliva sample which generate a reaction in a testing area may include a specific concentration of a particular compound in the saliva sample. Types of the particular compounds may include an enzyme, bacterium, virus, protein, or fungus. Examples may include Glucosyltransferase, Streptococcus mutans, lactobacillus, human papillomavirus (HPV 16/HPV 18), candida, porphyomonas gingivalis, C-reactive protein, matrix metalloproteinase-8 (MVIP-8), Interleukin-6 (IL-6), Atopobium parvulum, Eubacterium sulci, Fusobacterium periodonticum, and Solobacterium moorei. For example, one of the test areas of the testing device may generate a reaction in response to the presence or concentration of candida.

The attributes of the saliva sample which generate a reaction in a testing area may also include pH and/or buffering capacity of a saliva sample.

In at least one embodiment, a non-transitory computer readable medium includes computer readable instructions for execution on a computing device to cause a computing device to automatically analyze the image and reactions by the colorimetric analysis and/or lateral flow assay and display the results.

In at least one embodiment, a kit for use in conducting the above method includes the testing device. In an additional embodiment a kit for use in conducting the above method also includes a sterile collection device. In a further embodiment a kit for use in conducting the above method also includes a dropper.

The kit may also include at least one of: the above non-transitory computer readable medium, information for a user to access the above non-transitory computer readable medium, or a graphic for scanning or imaging to cause a computing device to access or download the above computer readable instructions and/or testing platform.

The above summary is to be understood as cumulative and inclusive. The above-described embodiments and features are combined in various combinations in whole or in part in one or more other embodiments.

### BRIEF DESCRIPTION OF THE DRAWINGS

The previous summary and the following detailed descriptions are to be read in view of the drawings, which illustrate particular exemplary embodiments and features as briefly described below. The summary and detailed descriptions, however, are not limited to only those embodiments and features explicitly illustrated.

FIG. 1 is an illustration of a kit for saliva sample testing, according to at least one embodiment;

FIG. 2 shows a saliva sample being delivered to a test area of a testing device, according to at least one embodiment;

FIG. 2A shows a testing device with a casing, according to at least one embodiment;

FIG. 3 shows the sample reactive device of FIG. 2 with reactions generated at several test areas in response to delivered saliva samples;

FIG. 4 shows a computing device having captured an image of the testing device of FIG. 3;

FIG. 5 shows an application or program, according to at least one embodiment, running on a user computing device;

FIG. 6 shows a test result window, according to at least one embodiment, displayed on a display module of the computing device of FIG. 4;

FIG. 7 shows a trend identifier, according to at least one embodiment, displayed on a display module of the computing device of FIG. 4;

FIG. 8 shows a flow chart indicating a generalized method, according to at least one embodiment, of utilizing a testing device to generate results; and

FIG. 9 shows a diagram of a user computing device in communication with a remote computing device through a network, according to at least one embodiment.

### DETAILED DESCRIPTIONS

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although steps may be expressly described or implied relating to features of processes or methods, no implication is made of any particular order or sequence among such expressed or implied steps unless an order or sequence is explicitly stated.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings. However, for each drawing, at least one embodiment is made according to the apparent relative scale of the drawing.

Like reference numbers used throughout the drawings depict like or similar elements. Unless described or implied as exclusive alternatives, features throughout the drawings and descriptions should be taken as cumulative, such that features expressly associated with some particular embodiments can be combined with other embodiments.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Generally, the terminology “testing platform” is utilized to refer to a specific application or program utilized within the context of this particular method. In various contexts, it is understood that “application or program” is interchangeable with “testing platform,” such as in the context of carrying out the steps of methods described herein on a computing device. For example, reference to the “application or program” analyzing an image, conducting calibration, or generating test results would also refer to the “testing platform” performing such functions.

According to the below described particular embodiments, with reference to the drawings, and those further embodiments and examples within the full scope of these descriptions and drawings, a user can self-administer a saliva sample test with a reactive testing device-having integrated colorimetric and lateral flow assay test areas-outside a health care facility, for example at home, and instead of having to collect urine or blood and/or send a sample into to a laboratory for analysis, capture an image of the testing device after the reactions which is provided to and analyzed by a testing platform to generate results, with the analysis and results based an algorithm and AI tech. These benefits, and those described hereafter, define features that are innovative in self-administered rapid results health testing.

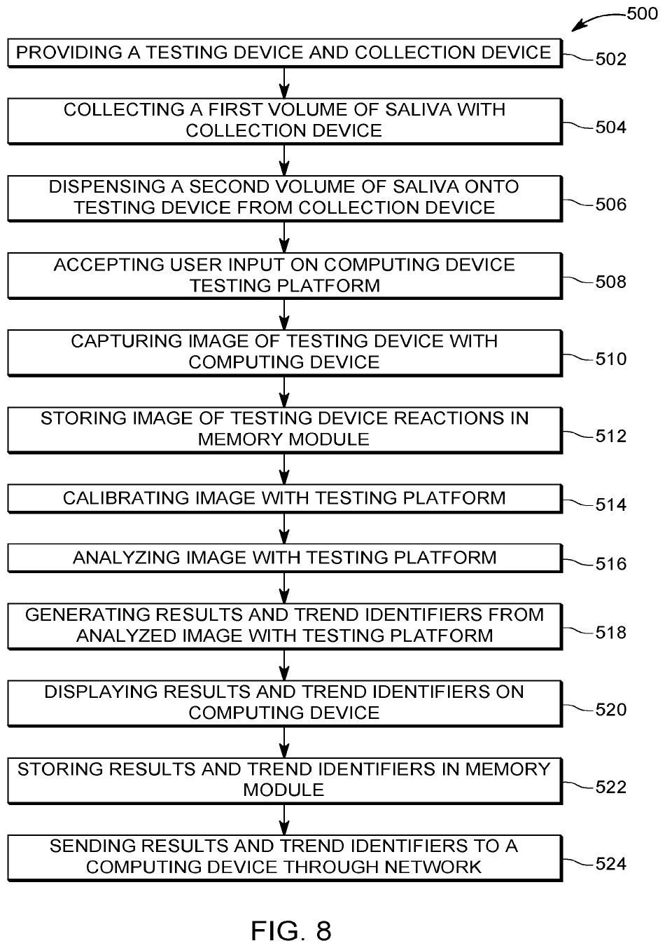

An overview of a general method 500 of utilizing the testing device, collection device, and testing platform according to at least one embodiment is provided in FIG. 8. That method 500 comprises the steps of providing a testing device and collection device 502; collecting a first volume of saliva with collection device 504; dispensing a second volume of saliva onto testing device from collection device 506; accepting user input on computing device testing platform 508; capturing image of testing device with computing device 510; storing image of testing device reactions in memory module 512; calibrating image with testing platform 514; analyzing image with testing platform 516; generating results and trends from analyzed image with testing platform 518; displaying results and trend indicators on computing device 520; storing results and trends in memory module 522; and sending results and trend indicators to a computing device through network 524. In order to better understand the above method and to highlight additional embodiments, elements, features, and steps are discussed in greater detail below.

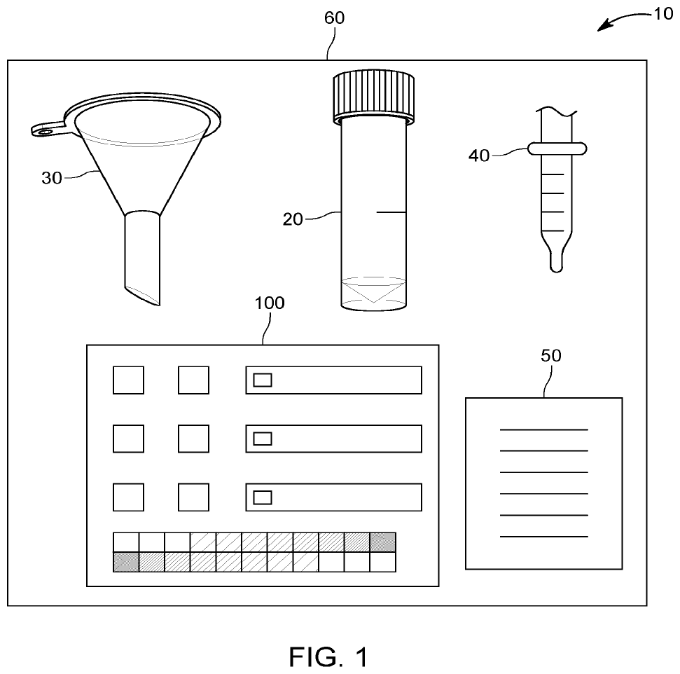

FIG. 1 shows a kit 10, according to at least one embodiment, for saliva sample testing at home or at other locations convenient to users. Testing need not be conducted at a dental or other health care facility. The kit 10, in the illustrated embodiment, includes items for saliva sample collection, preparation, and dispensing. A saliva testing device 100 is included to receive dispensed portions of a collected saliva sample at respective test areas of the device. The construction and layout of the testing device 100 can vary among embodiments thereof.

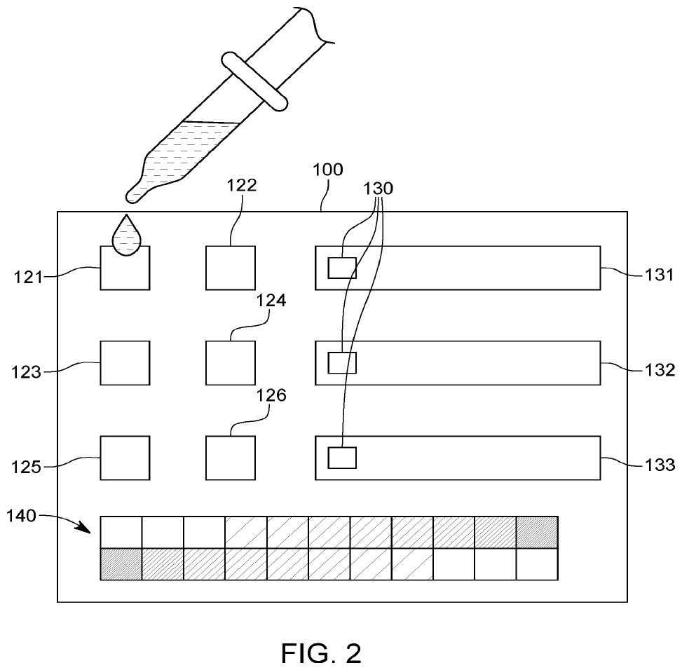

In the embodiment of FIG. 1, the testing device 100 has a planar card or board form upon which multiple colorimetric and lateral flow test areas are provided. The testing device 100 may be constructed, for example, of stiff absorbing paper or pressed fibrous material. As in FIG. 2A, the testing device 100 may also be constructed with a casing 110 surrounding the testing device and including openings in a surface thereof to allow a user to access and view the test areas 127, 134. For example, the testing device 100 may include a plastic casing 110 having dispensing portion 130 exposed through cutouts, the cutouts indicating where to dispense portions of the collected saliva sample and/or allowing a user to see the reactions within the testing areas 127, 134. A casing 110 may add to the rigidity, durability, and longevity of a testing device 100 and protect the test areas. Openings in the casing 110, such as those surrounding dispending portions 130 and testing areas 127 in FIG. 2A, may help direct dispensed samples into the respective test areas. Indeed, the casing 110 may prevent a saliva sample meant to be dispensed in one test area from inadvertently entering another test area, which may already have a sample. Cutouts in the casing 110 may help provide indications of which portions of the test area are dispensing portions by separating a dispensing portion 130 and the other portion where reactions will be generated 134, as shown in FIG. 2A.

In the embodiment of FIG. 1, each test area includes, or is itself marked by, a box to guide a user in delivering respective portions of a collected sample. These marked dispensing portions of the test area might represent all or just a portion of a respective test area. A respective color response may develop as prompted by the presence and attributes of a respective delivered portion of the sample at each colorimetric test area. A color response expresses the result of each attribute-based test. Thus, multiple colorimetric tests are conducted with the saliva testing device 100. Similarly, one or more respective indicators 150 (FIG. 2A) may develop as prompted by the presence and attributes of a respective delivered portion of the sample at each lateral flow assay (LFA) test area. The presence of the indicator 150 and/or its positioning within the test area expresses the results of one or more attribute-based tests, as in FIG. 2A. Thus, multiple LFA tests are conducted with the saliva testing device 100. The color responses and generated indicators are analyzed by a software application or program 400, also referred to as the testing platform herein, on a computing device 300, such as the user's phone, tablet, or laptop, by use of a color picture 200 taken of the testing device 100 after it has reacted with the dispensed samples. To conduct the multiple tests, a user dispenses a prescribed amount of saliva sample on each marked dispensing portion of the test area. These marked dispensing portions of the test area might encompass all or just a portion of a respective test area. For example, the dispensing portion comprises the entire colorimetric test areas 121-126, marked by the boxes, and the smaller respective boxes 130 of the LFA test areas 131-133 as referenced in FIG. 2. Note that, while boxes are shown, the dispensing portions might be indicated through markings of other shapes. Also, in FIG. 2A, dispensing portion 130 is for the LFA test area 134, which generates indicators 150 and, for the colorimetric test areas 127, the dispensing portion thereof encompasses all of the respective test area 127.

The kit 10, in the illustrated embodiment of FIG. 1, also includes a vial 20 into which a saliva sample can be delivered by a user. The vial 20 may have markings to indicate whether a sufficient prescribed amount of sample is collected in the vial. The vial 20 may also have markings to indicate a prescribed amount of sample to be dispensed in each dispensing portion. The kit 10 may further include additional collection aids such as a funnel 30 or cup. The kit 10, as illustrated in FIG. 1, also includes a dropper 40 for use by a user to deliver drops of saliva sample to the dispensing portions of test areas of the testing device 100. For example, a prescribed number of drops can be delivered to each test area from the vial 20 using the dropper 40. The dropper 40 may also include markings indicating a prescribed amount of sample to be dispensed for each sample, as in FIG. 1. Thereby, a user may fill a dropper and dispense the amount for each test through identification based on markings or by the numbers of drops. The vial 20, funnel 30, and dropper 40 may be sterile, in embodiments, to prevent contamination and/or inaccurate results. The use of the vial 20 and dropper 40, where a first volume of saliva is collected and a second volume is taken from the first to be dispensed in the test areas provides an efficient means to ensure samples are obtained under the same conditions. Thereby, the precision of the results might be enhanced.

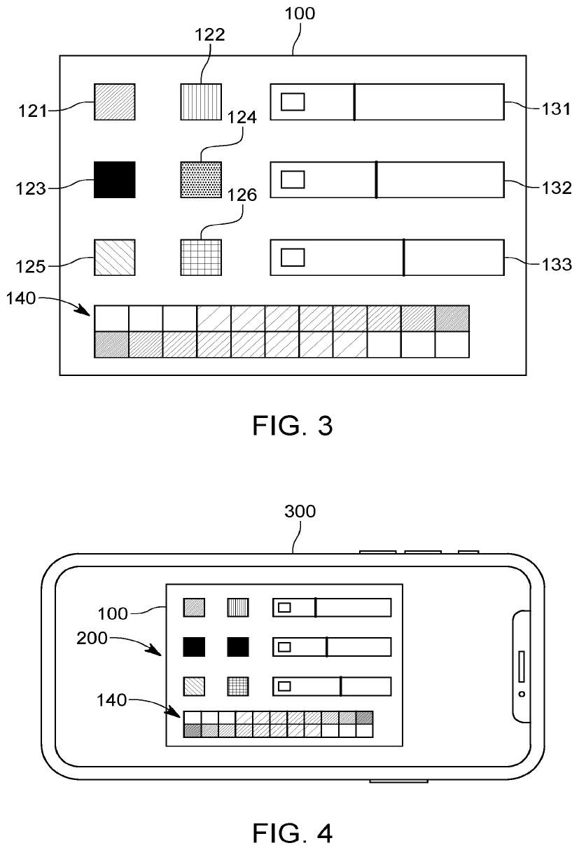

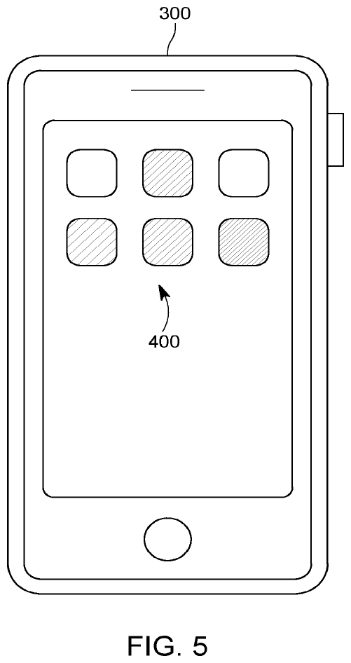

The kit 10 may further include instructions 50 in text and/or graphical form. The instructions 50 may be included upon the exterior or interior of a package 60, represented diagrammatically in FIG. 1 as a box. Instructions 50 may also, or alternatively, be included as an item in the kit 10. The instructions may include markings for scanning and automated information access, such as bar codes and QR codes, by which a computing device 300 (FIG. 4), such as a mobile phone or tablet, can access information, and/or download and update an application or program 400 (FIG. 5), such as the testing platform. The information and/or application 400, such as the testing platform, performs colorimetric and LFA analysis of images taken of the testing device 100 after an amount of saliva sample has been dispensed to the test areas and the reactions of each test area are complete.

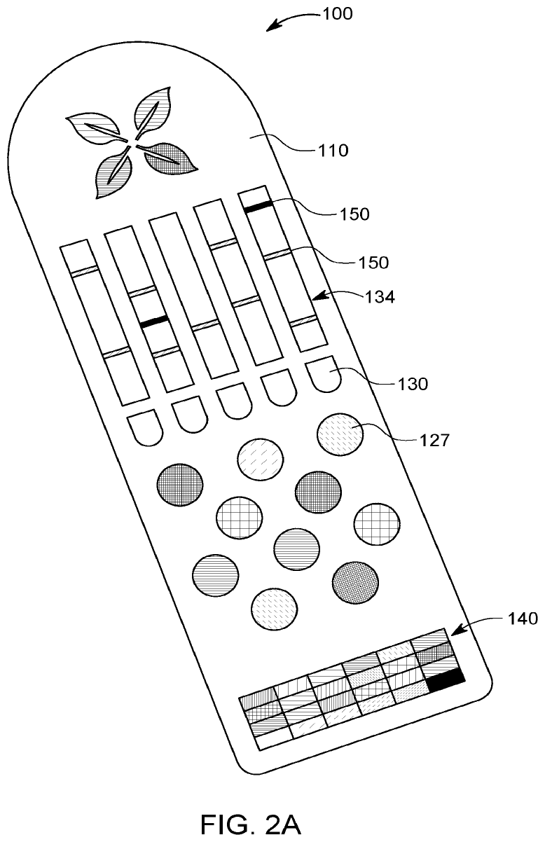

Regarding the use of the testing device 100, a respective test can be conducted at each of the areas 121-126, in each of which a result is evidenced by a change of color, for example by a color reagent sensitive to a particular biomarker, at the dispensing portion—where an amount of saliva sample is deposited by the user. FIG. 3 represents the discernible response of each test area 121-126 as a shade or pattern for purposes of distinction and description. This should be taken as a non-limiting example. In other examples within the scope of these descriptions and drawings, the response of each test area can appear as a shading, a coloration, a pattern, and/or any combination of these or other visible or optically discernible responses. Additionally, a respective test can be conducted at each of the areas 131-133 by lateral flow assay (LFA). Each test area 131-133 has a respective marked dispensing portion 130 (shown in FIG. 2) where a sample is to be delivered by a user. Lateral flow of the sample from the deposition spot causes a discernible response along or within a strip area of the respective test area. FIG. 3 shows the response of each test area 131-133 as a visible indicator band within the respective strip area. This should be taken as a non-limiting example. In other examples within the scope of these descriptions and drawings, the response of the strip area can appear as a shading, a coloration, a pattern, and/or any combination of these or other visible or optically discernible responses. Indeed, multiple test indicators and/or the location of generated indicators within the test areas may offer different discernible responses, allowing multiple tests to be performed on a single sample provided in a dispensing area of the testing device.

Particular non-limiting exemplary saliva-based tests that may be conducted by use of one or more of the colorimetric test areas 121-126 and LFA test areas 131-133 include tests for pH, buffering capacity, total ammonia, and the detections of various specific biomarkers, such as enzymes, bacteria, viruses, proteins, and fungi. Particular non-limiting examples of biomarkers discernable by the test areas include glucosyltransferase, Streptococcus mutans, lactobacillus, human papillomavirus (such as HPV 16 or 18), candida, Porphyromonas gingivalis, C-reactive protein, matrix metalloproteinase-8 (MMP-8), interleukin-6 (IL-6), Atopobium parvulum, Eubacterium sulci, Fusobacterium periodonticum, and Solobacterium moorei-one of more of which may be detectable using commercially available antibodies and/or color reagents. Alternatively, certain test areas may utilize specially developed antibodies or color reagents to react to attributes of the saliva sample, such as the presence and/or concentration of compounds or biomarkers. For example, the shading of a color change generated by a color reagent in a colorimetric test may be darker in the presence of a higher concentration of a particular compound, such as one of the biomarkers. In an additional example, the placement or number of indicators generated by an LFA test may result from the concentration of a particular compound.

Some latency may be expected for the color responses and/or indicators of the test areas to develop. Furthermore, accurate test results may rely upon prompt action after development of a color response or generation of an indicator. The user may be instructed as to the proper time window for action, e.g., the capture of an image 200 of the testing device 100, following complete distribution of the respective sample portions to the test areas. The instructions included with the kit 10 and/or information from the testing platform 400 may make user aware of any time windows for test area reactions. For example, the testing platform 400 may include instructions for a user to add saliva samples to dispensing portions of test areas in a particular order and/or timers to indicate when to distribute samples to dispensing portions and/or when to capture an image 200.

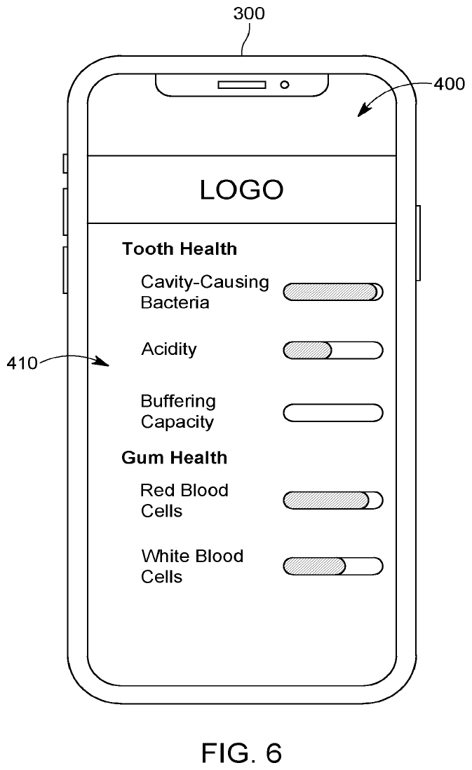

After a user has conducted the multiple tests, an image 200 of the testing device 100 is taken showing the generated responses of the test areas. For example, a user may take a color image 200 using his or her computing device 300, such as a tablet or mobile phone as represented in FIG. 4. The user activates a software application or program 400 (FIG. 5), such as the testing platform, which may for example be installed and running on the mobile device. The application or program 400 analyzes the image 200 of the developed sample reactive device 100 to generate results 410 (FIG. 6) based on the generated color changes and indicators from an algorithm and/or Al.

Preceding or otherwise in conjunction with the colorimetric analysis, in some examples, the application or program 400 conducts a colorimetric and/or LFA calibration by use of a color palette 140 provided on the testing device 100. The color palette 140 is represented in the drawings as a series of shaded boxes for purposes of distinction and description. The color palette 140 in implementation of the testing device 100 can include any arrangement or number of colored markings and indicators.

The application or program 400 conducts calibration using one or more separate images of the color palette 140, or the potion of the testing device image 200, showing the color palette 140 to measure, adjust, and/or compensate for the color response of the particular device 300 device, the state or settings of which may otherwise vary among devices, users, and user preferences. The calibration is thus used to normalize various devices and settings to assure accurate results from colorimetric and LFA analyses.

The application or program 400 further generates and displays test results 410 as represented in FIG. 6. The particular metrics represented in FIG. 6 are provided for example purposes only and are non-limiting. These and other metrics may be provided as results of colorimetric and LFA analyses of an image taken of the developed test areas of the testing device 100. In certain embodiments, the application or program 400 may also ask a user to input certain information or confirm they have taken certain actions to increase the accuracy of test results generated, such as part of the accepting user input step 508 in FIG. 8. For example, the user may have to confirm they have not ingested any food or drink item for a particular period which might influence the test. In some embodiments, the application or program 400 may prompt a user to input or confirm information before providing instructions or timers related to the testing or before capturing an image of the testing device 100, such as part of analyzing the image with the testing platform step 516 in FIG. 8. Instructions 50 may also provide information relating to actions which improve accuracy of the test results. The application or program 400 may further provide instructions related to the capture of the image, such as instructions related to the positioning of the imaging sensor of the computing device relative to the testing device or the lighting provided to illuminate the testing device. For example, the application or program 400 may instruct a user to move an imaging sensor closer to a testing device or utilize a flash feature to capture a better image of the testing device.

Through the use of the application or program 400, rapid user-location test results 410 are provided in embodiments. Thus, at-home saliva testing can be conducted to analyze one or more of pH, buffering capacity, total bacteria levels, Red Blood Cell (RBC) levels, White Blood Cell (WBC) levels, total protein levels, total gram-negative bacteria levels and total ammonia. Further tests that can also be conducted include the detection of glucosyltransferase, Streptococcus mutans, lactobacillus, human papillomavirus (such as HPV 16 or 18), candida, Porphyromonas gingivalis, C-reactive protein, matrix metalloproteinase-8 (MMP-8), interleukin-6 (IL-6), Atopobium parvulum, Eubacterium sulci, Fusobacterium periodonticum, and Solobacterium moorei. The tests results may be used to determine oral health parameters, providing insight into possible issues and solutions. For example, the test results may provide screening for potential oral cancer.

All tests, and test areas, may utilize one or more of colorimetric analysis or LFA. For example, the total bacteria level may be determined by a test area using colorimetric analysis, LFA analysis, or both. Further, it is foreseen that tests, and test areas may utilize combined LFA to provide additional variable detail to the results. For example, WBC levels may be tested with a single LFA, to determine total WBC levels, or with a combined LFA, to determine levels of various WBC types. In instances of combined LFA test areas, results may be generated by multiple indicators and/or their positioning within the test area, in certain embodiments. Moreover, the tests, and test areas, may utilize one or more specially crafted or commercially available antibodies or color reagents. For example, test areas for determining RBC levels may utilize commercially available anti-RBC antibodies with an LFA to provide test results.

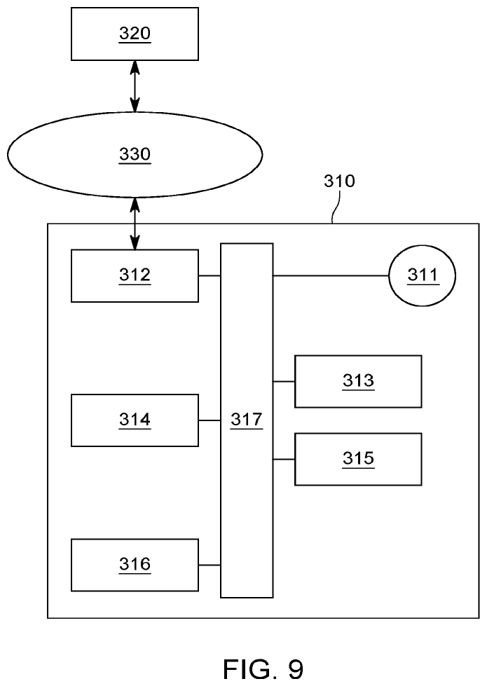

Use of the kit 10 with the application or program 400 provides quantitative indices through simple procedures as described herein that the patient can confidently conduct. Optionally, test results may be verified or further provided by additional analysis of the image 200 or metrics thereof by submission of the image 200 or related data to a central computing system, for example by upload across the internet or any suitable network or connectivity. Optionally, the application or program 400 may be web-based or provided on a separate computing device 320 accessed by a user's computing device 310 through a network 330, as in FIG. 9.

In embodiments, the user computing device 300 may include an imaging sensor 311, processing module 313, memory module 315, network communication module 312, user input module 314, and display module 316 electronically connected through at least one bus 317, as shown in FIG. 9. In additional embodiments, one or more of the components of the user computing device 310 may be externally electronically connected. In embodiments, the computing device 310 may be in communication with remote computing devices 320, such as a central computing system, through a network 330—as in FIG. 9. The remote computing devices 320 may also comprise one or more remote network accessible memory modules, such as a cloud-based server. In certain examples, the imaging sensors 311 of the user computing device 300 are used to capture an image 200 of the testing device 100. The captured image 200 may be stored in the memory module 316 of the computing device 300 and accessed by the application or program 400 during analysis. The user computing device 300 may include a network communication module 312 so that the image 200 and/or the results may be shared with a remote computing device 320, such as a central computing system, in particular embodiments.

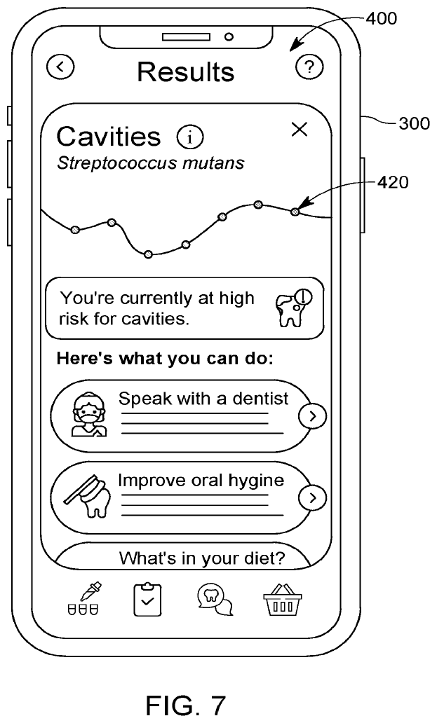

In embodiments, the user computing device 300 may access the application or program 400 from the memory module 315 of the user computing device 310 or through the network communication module 312, such as when the testing platform is stored in network accessible memory on a remote computing device 320. In embodiments, the test results 410 may include time and date data and be stored in the memory module 315 of the user computing device 310 or a remote computing device 320, such as through the network communication module 312. The application or program 400 may use the time and date data to generate trend identifiers 420, such as graphs or informational indicators, showing the progression of the results for a particular test, as in FIG. 7. The trend identifiers 420 may provide additional information to help determine oral health parameters, providing insight into possible issues and solutions-including the effectiveness of treatments. Additionally, the results 410, trend identifiers 420, and any other information and/or requests displayed to a user by the application or program 400 may be displayed on a display module 316 of the user computing device 310 in embodiments. The display module 316 may be integrated or separately electronically connected to the user computing device in embodiments.

The application or program 400, kit 10, and/or testing device 100 may be provided, in whole or in part, to consumers via online offer or sales and subsequent delivery or pick up, at a point-of-sale, and at a care facility such as a dentist's office. These should be taken as non-limiting examples. In at least one example, the kit 10 and/or testing device 100 are provided during, after, or before a visit to a dental health care facility. The application or program 400 can be provided with the kit 100 and/or device 100, for example on a non-transitory computer-readable medium by which the application or program 400 can be installed on a user computing device 310. The application or program 400 may be downloaded or otherwise acquired through the internet or any suitable network or connectivity. For example, download instructions may be included with the kit 10, such as by instructions 50, or online via an address provided with the kit 10 and/or testing device 100 itself. The kit 10, or testing device 100 itself, may include download instructions for manual use by the user or in the form of, for example, bar codes and/or QR codes by which a computing device, such as a tablet or mobile phone, can automatically access information, and/or download and update the application or program.

FIGS. 4-6 referencing a mobile device are also more generically a diagrammatic representation of a computing device. As shown in the example of FIG. 9, a computing device 300 includes components such as a processor 313, a storage device or memory 315. The network communication module 312 can comprise a communications controller that facilitates data input and output to a radio. The user input module 314 can include devices such as a keyboard or other buttons facilitate interface with a user. Examples of input devices include, but are not limited to, alphanumeric input devices, mice, electronic styluses, microphones, and scanners. In addition to the display module 316 of the computing device 300, the computing device 300 might also utilize one or more of signal generation devices (e.g., speakers) and printers to output information to a user. These devices may be internal or externally electronically connected with the user computing device 300. For example, a system bus 317 or other link interconnects the components of the computing device in embodiments. A power supply, which may be a battery or voltage device plugged into a wall or other outlet, powers the computing device 300 and its onboard components.

By way of example, and not limitation, the processor may be a general-purpose microprocessor such as a central processing unit (CPU), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and other manipulations of information.

The storage device or memory module 315 may include but is not limited to: volatile and non-volatile media such as cache, RAM, ROM, EPROM, EEPROM, FLASH memory or other solid state memory technology, disks or discs or other optical or magnetic storage devices, or any other medium that can be used to store computer readable instructions and which can be accessed by the processor. In at least one example, the storage device or memory module of the depicted computing device is or includes a non-transitory medium upon which computer readable instructions are stored. Upon the processor reading and executing the computer readable instructions, the depicted computing device conducts a method of colorimetric and LFA analysis of a color image as described herein and displays results for user viewing on a display as represented in FIG. 6.

Computer readable instructions may include program code, or instructions, for conducting operations for aspects of the present invention which may be written in any combination of one or more programming languages, including an object-oriented programming language such as Java, Smalltalk, C++, or the like and conventional procedural programming languages, such as the “C” programming language or similar programming languages. The program code may execute entirely on the user's computing device, partly on the user's computing device, as a stand-alone software package, partly on the user's computing device and partly on a remote computing device or entirely on the remote computing device, or server. In the latter situation scenario, the remote computing device may be connected to the user's computing device through any type of network, including a local area network (LAN) or a wide area network (WAN), or the connection may be made to an external computer (for example, through the Internet using an Internet Service Provider).

These computer readable instructions may also be stored in a computer readable medium that when executed can direct a computing device, other programmable data processing apparatus, or other devices to function in a particular manner, such that the instructions when stored in the computer readable medium produce an article of manufacture including instructions which when executed, to cause a computing device to implement the function/act specified in a flowchart and/or block diagram block or blocks. The computer readable instructions may also be loaded onto a computing device, other programmable instruction execution apparatus, or other devices to cause a series of operational steps to be performed on the computing device, other programmable apparatuses or other devices to produce a computer implemented process such that the instructions which execute on the computing device or other programmable apparatus provide processes for implementing the functions/acts specified in a flowchart and/or block diagram block or blocks.

As will be appreciated by one skilled in the art, aspects of the present disclosure may be illustrated and described herein in any of a number of patentable classes or contexts including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” “system,” or “unit,” in certain contexts. Furthermore, aspects of the present disclosure may take the form of a computer program product comprising one or more computer readable media having computer readable program code embodied thereon.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present inventive subject matter. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

It will be understood that when an element or layer is referred to as being “on” another element or layer, the element or layer can be directly on another element or layer, or intervening elements or layers may also be present. In contrast, when an element is referred to as being “directly on” another element or layer, there are no intervening elements or layers present. As used herein, the term “and/or” includes any and all combinations of one or more of the associated listed items.

Embodiments of the inventive subject matter are described herein with reference to plan and perspective illustrations that are schematic illustrations of idealized embodiments of the inventive subject matter. As such, variations from the shapes of the illustrations as a result, for example, of manufacturing techniques and/or tolerances, are to be expected. Thus, the inventive subject matter should not be construed as limited to the particular shapes of objects illustrated herein, but should include deviations in shapes that result, for example, from manufacturing. Thus, the objects illustrated in the figures are schematic in nature and their shapes are not intended to illustrate the actual shape of a region of a device and are not intended to limit the scope of the inventive subject matter.

The terminology used herein is for the purpose of describing particular embodiments only and is not intended to be limiting of the present inventive subject matter. As used herein, the singular forms “a,” “an” and “the” are intended to include the plural forms as well, unless the context clearly indicates otherwise. It will be further understood that the terms “comprises” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this present inventive subject matter belongs. It will be further understood that terms used herein should be interpreted as having a meaning that is consistent with their meaning in the context of this specification and the relevant art and will not be interpreted in an idealized or overly formal sense unless expressly so defined herein. The terms “plurality” and “multiple” are used herein to refer to two or more of the referenced items. Although any methods, devices, and materials similar or equivalent to those described herein can be used in the practice or testing of the presently disclosed subject matter, representative methods, devices, and materials are now described.

In the drawings and specification, there have been disclosed typical preferred embodiments of the inventive subject matter and, although specific terms are employed, they are used in a generic and descriptive sense only and not for purposes of limitation, the scope of the inventive subject matter being set forth in the following claims.

Particular embodiments and features have been described with reference to the drawings. The description of the present invention and particular embodiments and features has been presented for purposes of illustration and description but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated. It is to be understood that these descriptions are not limited to any single embodiment or any particular set of features, and that similar embodiments and features may arise or modifications and additions may be made without departing from the scope of these descriptions and the spirit of the appended claims.

1-18. (canceled)

19. A saliva test kit comprising:
a saliva sample container for holding collected saliva;
a dropper for selectively conveying a limited volume of at least a portion of the collected saliva;
a saliva testing device, the testing device comprising
a substantially planar test platform;
a first array of saliva testing areas, the first array of saliva testing areas being colorimetric analysis test areas;
a second array of saliva testing areas adjacent the first array of saliva testing areas, the second array of saliva testing areas being flow assay test areas on which saliva composition visible indicators may develop;
a layer underlying the first and second arrays composed of a fluid-absorbing material;
a color pallet adjacent the first and second arrays of saliva testing areas; and
a computing device operatively associated with the saliva testing device for generating one or both of a display selected from the group consisting of test results and trend identifiers of the saliva testing areas and assessing and reporting saliva analysis test results.

20. The saliva test kit of claim 19 wherein said computing device includes an imaging device for capturing an image of the saliva testing areas.

21. The saliva test kit of claim 19 wherein the computing device includes a test result readout.

22. The saliva test kit of claim 21 wherein the test result readout includes tooth health test results selected from the group consisting of bacteria levels, acidity level, and buffering capacity.

23. The saliva test kit of claim 19 wherein the computing device includes a memory module for storing test results and test assessments.

24. A saliva test kit for identifying health trends comprising:
a saliva sample container for holding collected saliva;
a dropper for selectively conveying a predetermined volume of the collected saliva;
a saliva testing device, the testing device comprising
a substantially planar test platform;
a first array of saliva testing areas;
a second array of saliva testing areas adjacent the first array of saliva testing areas;
a trend identifier selected from the group consisting of graphs and information indicators, the trend identifier providing data selected from the group consisting of one or more tests, oral health parameters, risk levels, and treatment effectiveness;
a color pallet adjacent the first and second arrays of saliva testing areas; and
a computing device operatively associated with the saliva testing device for reading the results of the saliva testing areas and assessing and reporting saliva analysis test results.

25. The saliva test kit of claim 24 wherein said first array of saliva testing areas comprises colorimetric analysis test areas.

26. The saliva test kit of claim 24 wherein said second array of saliva testing areas comprises flow assay test areas.

27. The saliva test kit of claim 24 wherein said computing device includes an imaging device for capturing an image of the saliva testing areas.

28. The saliva test kit of claim 24 wherein the computing device includes a test result readout.

29. The saliva test kit of claim 28 wherein the test result readout includes tooth health test results selected from the group consisting of bacteria levels, acidity level, and buffering capacity.

30. The saliva test kit of claim 28 wherein the computing device includes a testing platform for analyzing the test results following reaction of the of test areas to the saliva.

31. The saliva test kit of claim 24 wherein the computing device includes a memory module for storing test results and test assessments.

32. A self-contained saliva testing apparatus comprising:
a substantially planar test platform having a testing array surface;
a first array of saliva testing areas formed on the testing array surface, the first array of saliva testing areas being colorimetric analysis test areas, the first array of saliva testing areas being formed in a restricted saliva test responsive area including a reagent responsive to one or more biomarkers;
a second array of saliva testing areas formed on the testing array surface adjacent the first array of saliva testing areas, the second array of saliva testing areas being flow assay test areas, the second array of saliva testing areas being formed in one or more band-indicating strips of test responsive areas and including a reagent responsive to one or more biomarkers;
a layer underlying the first and second arrays composed of a fluid-absorbing material; and
a color pallet formed on the testing array surface adjacent the first and second arrays of saliva testing areas.

33. The self-contained saliva testing apparatus of claim 32 wherein the reagents of the first and second arrays respond to biomarkers according to the indictors selected from the group consisting of changes in shading, changes in color, and the appearance of an image pattern.

34. The self-contained saliva testing apparatus of claim 32 wherein the first and second arrays include tests selected from the group consisting of pH, buffering capacity, and ammonia volume.

35. The self-contained saliva testing apparatus of claim 32 wherein the first and second arrays detect biomarkers from the group consisting of enzymes, bacteria, viruses, proteins, and fungi.

36. The self-contained saliva testing apparatus of claim 32 wherein at least one of the first and second arrays detect biomarkers selected from the group consisting of glucosyltransferase, Streptococcus mutans, lactobacillus, human papillomavirus (HPV 16/HPV 18), Candida, porphyomonas gingivalis, C-reactive protein, matrix metalloproteinase-8 (MMP-8), Interleukin-6 (IL-6), Atopobium parvulum, Eubacterium sulci, Fusobacterium periodonticum, and Solobacterium moorei.

37. The self-contained saliva testing apparatus of claim 32 wherein the first and second arrays indicate a response to one or more antibodies.

38. The self-contained saliva testing apparatus of claim 32 wherein the biomarkers are directed to disease states taken from the group consisting essentially of cavities, oral cancer, periodontal disease, and halitosis.

39. The self-contained saliva testing apparatus of claim 32 further including a casing for housing the test platform, the casing including one or more openings formed therein for directing the saliva samples to the test areas.

40. The self-contained saliva testing apparatus of claim 39 wherein the casing includes further includes a saliva-receiving opening formed in proximate relationship to each of the band-indicating strips.
